(19) United States
(12) Patent Application Publication
(10) Pub. No.: US 2006/0293331 A1
(43) Pub. Date: Dec. 28, 2006

(54) Process of synthesizing tadalafil

(76) Inventors: Ben-Zion Dolitzky, Petach Tiqva (IL); Dov Diller, Jerusalem (IL)

Correspondence Address: KENYON & KENYON LLP, ONE BROADWAY, NEW YORK, NY, 10004, US

(21) Appl. No.: 11/364,598

(22) Filed: Feb. 27, 2006

Related U.S. Application Data
(60) Provisional application No. 60/656,664, filed on Feb. 25, 2005.
(60) Provisional application No. 60/736,807, filed on Nov. 14, 2005.
(60) Provisional application No. 60/737,080, filed on Nov. 15, 2005.

(51) Int. Cl. A61K 31/498; C07D 487/14

(52) U.S. Cl. 514/249; 544/343

(57) ABSTRACT

The present invention provides tadalafil of high purity and processes of making such tadalafil by cyclization of TDCl in a solution.

### RELATED APPLICATIONS

This application claims the benefit of U.S. Provisional Application No. 60/656,664, filed Feb. 25, 2005; U.S. Provisional Application No. 60/736,807, filed Nov. 14, 2005; and U.S. Provisional Application No. 60/737,080, filed Nov. 15, 2005. The contents of these applications are incorporated herein by reference.

### FIELD OF THE INVENTION

The invention is directed to an improved synthesis of tadalafil by cyclization of TDCl in a solution.

### BACKGROUND OF THE INVENTION

Tadalafil, (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, with the structural formula shown below, is a white crystalline powder. (CAS# 171596-29-5). Tadalafil is a potent and selective inhibitor of the cyclic guanosine monophosphate (cGMP)-specific phosphodiesterase enzyme, PDE5. The inhibition of PDE5 increases the amount of cGMP, resulting in smooth muscle relaxation and increased blood flow. Tadalafil is therefore currently used in the treatment of male erectile dysfunction.

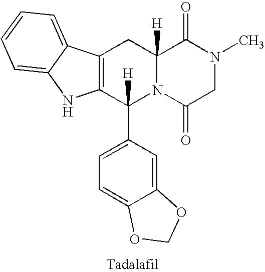

Current methods of synthesizing tadafil involve the cyclization of the tadalafil intermediate, TDCl, with methylamine.

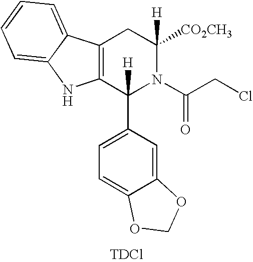

U.S. Pat. No. 5,859,006 describes the synthesis of tadalafil via the cyclization of TDCl using methylamine in a methanol slurry, with subsequent purification by flash chromatography. The cyclization reaction is typically performed in a slurry because TDCl has very poor solubility in organic solvents. Processes of producing tadalafil in a slurry require additional purification steps, such as multiple extractions, HPLC and the use of HCL, to remove the impurities that remain in the slurry after the synthesis of tadalafil is complete. Additional purification steps increase the cost of producing tadalafil.

WO 04/011463 describes a process of preparing tadalafil by the cyclization of TDCl in a solution of THF using methylamine. The process of producing tadalafil in a solution of THF requires using expensive and unsafe reagents.

There is a need in the art for improved methods of preparing tadalafil by cyclization of TDCl in a solution.

### SUMMARY OF THE INVENTION

The present invention provides a process of preparing tadalafil comprising the steps of: providing a solution of TDCl in a medium capacity reaction solvent, combining the solution with methylamine to form a reaction mixture, heating the reaction mixture and recovering tadalafil.

The present invention also provides tadalafil containing about 200-500 ppm or less chloride, as chloride ion.

### BRIEF DESCRIPTION OF THE FIGURES

FIG. 1 is a representative HPLC chromatogram, recorded as described below, for tadalafil made by a preferred embodiment of the present invention.

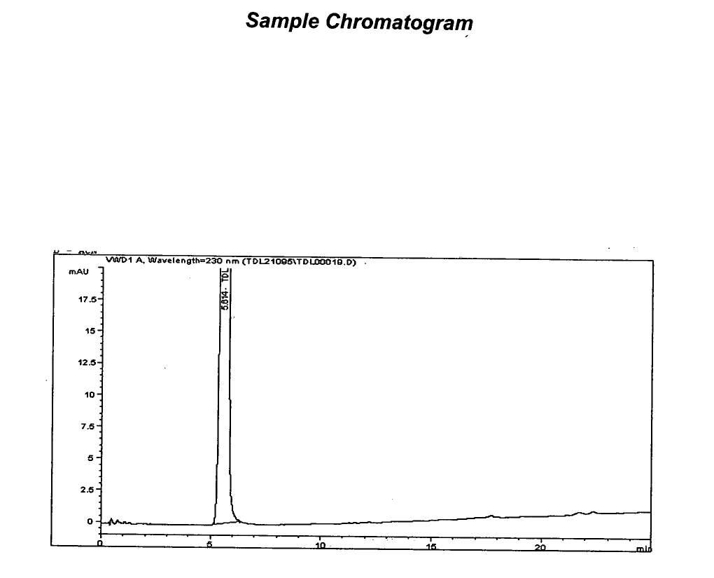

### DETAILED DESCRIPTION OF THE INVENTION

As used herein, “TDCl” refers to cis-methyl 1,2,3,4-tetrahydro-2-chloroacetyl-1-(3,4-methylenedioxyphenyl)-9H-pyrido[3,4-b]indole-3-carboxylate.

As used herein, “medium capacity reaction solvent” and “medium capacity solvent” refer to an organic solvent in which 1 gram of TDCl is soluble in about 10 ml to about 60 ml of the organic solvent. Medium capacity reaction solvents suitable for the process of the invention include nitrites, aromatic hydrocarbons, lower aliphatic alcohols, and especially alkyl esters of lower carboxylic acids.

The term “nitrites” refers to an organic compound having a —CN functional group. Acetonitrile is a preferred nitrile for use in the process of the invention.

The term “aromatic hydrocarbons” refers to C₆-C₁₀ monocyclic and polycyclic aromatic or substituted hydrocarbons including benzene, toluene, and tetrahydronapthalene, optionally having a C₆-C₁₂ alkyl, halo, or nitro substituent, and mixtures of these. Toluene is a preferred aromatic hydrocarbon for use in the practice of the present invention.

The term “lower aliphatic alcohols”, as used herein, refers to organic compounds having the general structure R—OH, wherein R is a linear or branched C₂₋₆ alkyl group. Lower aliphatic alcohols that are preferred for use in the process of the invention include ethanol, propanol, and butanol.

The term “alkyl esters of lower carboxylic acids,” as used herein, refers to organic compounds having the general structure R′—COOR″, wherein R′ is a linear or branched alkyl group having from 1 to 4 carbon atoms, and R″ is a linear or branched akyl group having from 1 to 6 carbon atoms. Preferred alkyl esters of lower carboxylic acids include ethyl acetate, propyl acetate, butyl acetate, isopropyl acetate, and isobutyl acetate. Particularly preferred alkyl esters of lower carboxylic acids are butyl acetate and isobutyl acetate.

The invention provides a process of synthesizing tadalafil that includes the cyclization of the intermediate TDCl with methylamine in a solution. The solution contains an inexpensive and safe medium capacity reaction solvent. The tadalafil obtained by this process is the trans isomer of tadalafil. The tadalafil synthesized in the process of the invention is easily isolated, and impurities may be removed from the solution in a cost efficient manner.

In one embodiment, the present invention provides a process comprising the steps of: providing a solution of TDCl in a medium capacity reaction solvent, combining the solution with methylamine to form a reaction mixture, heating the reaction mixture, and recovering tadalafil.

Preferably, the medium capacity reaction solvent is selected from the group consisting of: toluene, ethanol, propanol, butanol, acetonitrile, butyl acetate, propyl acetate, isobutyl acetate, and ethyl acetate. Most preferably, the medium capacity reaction solvent is isobutyl acetate or butyl acetate

Methylamine used in the process of the invention is provided as gas or as a solution in an organic solvent, preferably an alcohol such as ethanol, and is present in an amount sufficient to react with TDCl, for example, in a stoichiometric amount. Preferably, methylamine is present in an amount of about 2.5 to about 5 mol equivalents to TDCl.

Preferably, the reaction mixture is heated to a temperature of about 50° C. to about reflux temperature of the medium capacity reaction solvent. Preferably, the heating is for about 1 to about 48 hours, more preferably for about 1 to about 24 hours. The heating time depends on, among other things, the scale of the reaction, the size of the equipment used in the reaction, and the type of agitation provided, and can be determined by one skilled in the art by measuring the absence of the limiting reagent using such techniques as HPLC or TLC (thin layer chromatography).

The process of the invention can be performed in an open or closed reaction vessel. Preferably, the process of the invention is performed in a closed reaction vessel at a pressure of about 1 to about 2.5 atmospheres.

The process of the invention optionally comprises filtration. Filtration can be performed before combining the solution of TDCl with methylamine and/or after combining the solution of TDCl with methylamine, provided filtration is performed before any substantial precipitation of tadalafil occurs. Filtration, when performed, is preferably performed before combining the solution of TDCl with methylamine.

The process of the invention optionally comprises the step of cooling prior to filtration. The process also optionally comprises the step of washing tadalafil with at least one of medium capacity reaction solvent, water, or methanol.

Tadalafil can be recovered by any method known in the art, such as by slurrying with methanol and drying the precipitate.

When either n- or iso-butyl acetate, acetonitrile or toluene are used as the medium capacity reaction solvents, the tadalafil obtained has a level of purity such that the total level of impurities measured is less than about 0.5% area by HPLC and the level of any individual impurity is less than about 0.1% area by HPLC. Tadalafil obtained by the process of the invention preferably contains about 200-500 ppm or less chloride, as chloride ion.

The purity data for tadalafil disclosed herein were determined by high pressure liquid chromatography according to the method described below. The phrase “substantially free of”, i.e., substantially free of impurities, denotes that the total level of impurities in a tadalafil sample was undetectable by this HPLC method.

The present invention, in certain of its embodiments, is illustrated by the following non-limiting examples.

### EXAMPLES

HPLC Method:

| Column & Packing: | Zorbax SB-Phenyl 75 mm * 4.6 mm, 3.5μ | | |
|---|---|---|---|
| Buffer: | 0.02M of Sodium di-Hydrogen Phosphate | | |
| | Monohydrate (NaH₂PO₄) as is (pH about 4.5) | | |
| Eluent A; | Buffer | | |
| Eluent B: | Acetonitrile | | |
| Gradient: | Time | % Eluent A | % Eluent B |
| | 0 | 70 | 30 |
| | 6 | 70 | 30 |
| | 25 | 42 | 58 |

| | |
|---|---|
| Equilibrium time: | 3 min |
| Sample volume: | 10 μL |
| Flow Rate: | 2.0 mL/min. |
| Detector: | 230 nm |
| Column temperature: | 25° C. |
| Sample temperature: | 15° C. |
| Diluent: | 80% Acetonitrile, 20% Water |

The level of the chloride ion disclosed herein was determined by AgNO₃ titration.

Standard Solution Preparation

Accurately weigh about 16 mg of Tadalafil standard into a 20 mL volumetric flask. Fill the flask to volume with diluent and dissolve the tadalafil completely using a sonicator. Transfer 5.0 mL of the solution obtained into a 100 mL volumetric flask and fill it to volume with diluent. Transfer 2.0 mL of this solution (“Solution B”) into a 100 mL volumetric flask and fill it to volume with diluent (0.1%, 0.0008 mg/mL).

Sample Solution Preparation

Weigh accurately 16 mg of sample into a 20 mL volumetric flask. Dissolve the sample in diluent ultrasonically and fill the flask to volume with diluent (0.8 mg/mL).

Procedure

Inject the standard and sample solutions. Determine the areas of each peak using a suitable integrator.
Calculations

$$\% Imp . i = \frac{Area Imp . i}{Average response factor of TDL std \times Sample concentration} \times Potency TDL std$$

### Example 1

### Synthesis of Tadalafil in Acetonitrile

TDCl (5 g, 11.7 mmol), acetonitrile (125 ml), and methylamine (4 ml, 8 M, 32 mmol, 33% EtOH) were combined to form a reaction mixture. The reaction mixture was stirred and heated at about 90° C. for about four hours in an autoclave. The reaction mixture was cooled to room temperature and filtered. A solid was obtained, which was dried (3.43 g; 75% yield). The solid was determined to be tadalafil, with a purity of 100%, by HPLC.

### Example 2

### Synthesis of Tadalafil in Ethanol

TDCl (5 g, 11.7 mmol), ethanol (125 ml), and methylamine (4 ml, 8 M, 32 mmol, 33% EtOH) were combined to form a reaction mixture. The reaction mixture was stirred and heated at about 78° C. for about 5.5 hours in an autoclave. The reaction mixture was cooled to room temperature and filtered. A solid was obtained and dried (3.3 g; 72% yield). The solid was determined to be tadalafil, with a purity of 99.63%, by HPLC.

### Example 3

### Synthesis of Tadalafil in Toluene

TDCl (10 g, 23.4 mmol), toluene (200 ml), and methylamine (23 ml, 8 M, 184 mmol, 33% EtOH) were combined to form a reaction mixture. The reaction mixture was stirred and heated at about 89° C. for about six hours in an autoclave. The reaction mixture was cooled to room temperature and filtered. A solid was obtained, and was slurried twice with methanol (25 ml). The solid was then dried (7.8 g; 85.3% yield), and determined to be tadalafil, with a purity of 100%, by HPLC. The solid was free of chloride ions.

### Example 4

### Synthesis of Tadalafil in Butanol

TDCl (5 g, 11.7 mmol), n-butanol (180 ml), and methylamine (4 ml, 8 M, 32 mmol, 33% EtOH) were combined to form a reaction mixture. The reaction mixture was stirred and heated at about 84° C. overnight in an autoclave. The reaction mixture was cooled in an ice bath and filtered. A solid was obtained, which was dried (3.4 g; 75% yield). The solid was determined to be tadalafil, with a purity of 99.69%, by HPLC. The solid was free of chloride ions.

### Example 5

### Synthesis of Tadalafil in Isobutyl Acetate

TDCl (10 g, 23.4 mmol), isobutylacetate (250 ml), and methylamine (8.8 ml, 8 M, 70 mmol, 33% EtOH) were combined in a 500 ml, 3 neck flask equipped with a magnetic stirrer, thermometer, and condenser connected to a paraffin trap in an oil bath, to form a reaction mixture. The reaction mixture was stirred and heated at about 40° C. for about 24 hours. The reaction mixture was cooled in an ice bath and filtered. A solid was obtained, and was slurried twice with methanol (25 ml). The solid was dried (8.22 g, 90% yield) and determined to be tadalafil with a purity of 99.93%.

### Example 6

### Synthesis of Tadalafil in Ethyl Acetate

Tadalafil is synthesized in a solution of ethyl acetate and TDCl according to the following general procedure. A solution is formed by dissolving TDCl (1 mol equivalent) in a volume of ethyl acetate (25 ml/g TDCl) while heating. The solution is filtered. The solution is cooled to a temperature of about 20° C. to about 90° C., and methylamine (3 mol equivalents, 33% EtOH) is added, forming a reaction mixture. The reaction mixture is maintained at the given temperature until the reaction is complete, as measured by less than 0.5% TDCl remaining in the reaction mixture. The reaction mixture is then cooled in an ice bath and filtered. A solid is obtained, and is slurried twice with methanol (2.5 ml/g TDCl). The solid is dried, and tadalafil with a purity of 99.89-100% purity is obtained.

### Example 6(a)

The solution of TDCl in ethyl acetate is cooled to about 20° C., and methylamine is added, forming a reaction mixture. The reaction mixture is maintained at about 20° C. for about 2 days, and then cooled and filtered. A solid is obtained, and slurried with methanol twice. The solid is dried, yielding tadalafil substantially free of impurities.

### Example 6(b)

The solution of TDCl in ethyl acetate is cooled to about 90° C., and methylamine is added, forming a reaction mixture. The reaction mixture is maintained at about 90° C. for about 6 hours, and then cooled and filtered. A solid is obtained, and slurried with methanol twice. The solid is dried, yielding tadalafil substantially free of impurities.

### Example 7

### Synthesis of Tadalafil in Butyl Acetate

Tadalafil is synthesized in a solution of butyl acetate according to the following general procedure. A reaction mixture is formed by combining TDCl (1 mol equivalent), butylacetate (20 ml/g TDCl), and methylamine (3 mol equivalents, 8 M, 70 mmol, 33% EtOH) in an open reactor connected to a paraffin bubbler and equipped with an agitator. The reaction mixture is stirred at a temperature of about 20° C. to about 60° C. The reaction is stopped by cooling the reaction mixture to a temperature less than 5° C. when the amount of TDCl measures <0.5% of the reaction mixture. The reaction mixture is filtered and a solid is obtained. The solid is slurried twice in methanol (2.5 ml/g TDCl), and dried (87-90% yield). The tadalafil obtained has a purity of 99.9%.

### Example 7(a)

The reaction mixture is stirred at a temperature of about 20° C. for about two days. The reaction mixture is cooled to a temperature less than 5° C. and filtered. A solid is obtained and is slurried twice in methanol, then dried, yielding tadalafil substantially free of impurities.

### Example 7(b)

The reaction mixture is stirred at a temperature of about 60° C. for about ten hours. The reaction mixture is cooled to a temperature less than 5° C. and filtered. A solid is obtained and is slurried twice in methanol, then dried, yielding tadalafil substantially free of impurities.

What is claimed is:

1. A process for synthesizing tadalafil comprising:
a) providing a solution of TDCl in a medium capacity reaction solvent;
b) combining the solution with methylamine to form a reaction mixture;
c) heating the reaction mixture to obtain tadalafil; and
d) recovering tadalafil.

2. The process of claim 1, wherein the medium capacity reaction solvent is selected from the group consisting of nitrites, C₆-C₁₀ aromatic hydrocarbons, C₂-C₆ alcohols and alkyl esters of lower carboxylic acids.

3. The process of claim 2, wherein the medium capacity reaction solvent is selected from the group consisting of toluene, ethanol, propanol, butanol, acetonitrile, ethyl acetate, butyl acetate, propyl acetate, isopropyl acetate, and isobutyl acetate.

4. The process of claim 3, wherein the medium capacity reaction solvent is butyl acetate or isobutyl acetate.

5. The process of claim 1, wherein the reaction mixture is heated to a temperature of about 50° C. to about reflux temperature of the medium capacity reaction solvent.

6. The process of claim 5, wherein the reaction mixture is heated for about 1 to about 48 hours.

7. The process of claim 6, wherein the reaction mixture is heated for about 1 to about 24 hours.

8. The process of claim 1, wherein the process is performed in a closed reaction vessel at a pressure of about 1 to about 2.5 atmospheres.

9. The process of claim 1, further comprising the step of filtering the solution of step a) or the reaction mixture of step b).

10. The process of claim 9, wherein the solution of step a) is filtered.

11. The process of claim 10, further comprising the step of cooling the reaction mixture after the heating in step c).

12. The process of claim 1, wherein the methylamine is gaseous.

13. The process of claim 1, wherein the methylamine is in a solution with an organic solvent.

14. The process of claim 13, wherein the methylamine is in a solution with alcohol.

15. The process of claim 1, wherein the methylamine is present in an amount of about 2.5 to about 5 mol equivalents to TDCl.

16. The process of claim 1, wherein the tadalafil is recovered by washing with at least one of medium capacity reaction solvent, water or methanol.

17. Tadalafil obtained by the process of claim 1 having a level of impurity less than about 0.5% area by HPLC of total impurities.

18. The tadalafil of claim 17, wherein the level of any individual impurity is less than about 0.1% area by HPLC.

19. Tadalafil obtained by the process of claim 1 containing about 200-500 ppm or less chloride, as chloride ion.
